(19) United States
(12) Patent Application Publication
(10) Pub. No.: US 2005/0191264 A1
(43) Pub. Date: Sep. 1, 2005

(54) Gel-type styling agent containing pregelatinized, cross-linked starch derivatives

(76) Inventors: Marion Detert, Hamburg (DE); Anke Pilzner, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/511,124

(22) PCT Filed: Apr. 9, 2003
(86) PCT No.: PCT/EP03/03678

§ 371 Date: May 3, 2005

(30) Foreign Application Priority Data
Apr. 11, 2002 (DE) 10216499.1

(51) Int. Cl. A61K 7/06; A61K 7/11

(52) U.S. Cl. 424/70.13; 424/70.16

(57) ABSTRACT

The invention provides cosmetic hairstyling preparations exhibiting improved properties, such as improved behavior upon combing out of hair. The preparations comprise one or more pregelatinized, crosslinked starch derivatives, anionic polymers, nonionic polymers, and water. In one embodiment, the preparations are gel-type hair styling agents.

The present invention relates to cosmetic hairstyling preparations or styling agents, in particular gel-type hairstyling preparations, with improved properties, in particular with improved behavior upon combing the product out of the hair.

The entire human body, with the exception of the lips, the palms of the hands and the soles of the feet, is covered in hair, for the large part, however, with barely visible down. Because of the many nerve endings at the hair root, hair reacts sensitively to external influences such as wind or touch and is therefore a component of the sense of touch that should not be underestimated. The most important function of human head hair must, however, nowadays consist in helping to create the appearance of the person in a characteristic manner. Similarly to the skin, it fulfils a social function since, via its outward appearance, it contributes considerably to interpersonal relations and to the self-esteem of the individual.

The hair consists of the hair shaft which protrudes freely from the skin—the keratinized (dead) section which represents the actual visible hair—and the hair root which sticks in the skin—the living section, in which the visible hair is continually renewed. The hair shaft in turn is made up of three layers: a central section—the so-called hair marrow (medulla), which, however, in humans has retrogressed and is often missing altogether—also the marrow (cortex) and the external, horny layer up to ten layers thick (cuticle), which surrounds the entire hair.

Provided there are no pathological changes, it is virtually impossible to improve upon human hair in its freshly grown state. The section of a hair in the vicinity of the scalp accordingly has a virtually closed horny layer. In particular, the horny layer, being the external sheath of the hair, but also the inner region below the cuticle, are exposed to particular stress by environmental influences.

One aim of hair care is to protect the scalp and head hair from harmful influences and to maintain the natural condition of freshly grown hair over the longest period possible and, if it is lost, to restore it. Silky sheen, low porosity, and a pleasant smooth feel are features of natural healthy hair.

Since the end of the previous century products for hair care have been developed specifically. This led to a large number of preparations both for general hair care and also for alleviating the anomalies of hair and of the scalp. In general, use is nowadays made of hair care cosmetics which are, after they have worked, either intended to be washed out of the hair again, or which should remain on the hair. The latter can be formulated such that they not only serve to care for the individual hair, but also improve the appearance of a hair style overall, for example by giving the hair more fullness, fixing the hair style over a prolonged period or improving the stylability.

The invention provides styling agents and hair setting compositions based on water. An essential functional constituent of such preparations are film formers. As such, use is made of various polymers, for example mixed polymers or copolymers of vinyl acetate and vinlypyrrolidone and also anionic polymers or preferably combinations of anionic polymers with nonionic or cationic polymers. Such preparations are usually supplied for use in various forms, in particular as hair spray, styling gel, setting foam or hair lacquer. The styling agents are topical preparations and can also serve for the treatment and care of the scalp and/or of the hair or as photoprotective preparation.

Gels are aqueous or alcoholic/aqueous preparations which, besides the setting polymers, comprise anionic polymers as thickeners. The thickeners used are often polyacrylic acids, such as, for example, (INCI) Carbomers. After applying styling gels to the hair, a polymer film forms around the individual hairs or the sections of hair. This film tears upon combing the product out of the hair and leaves behind small white residues or particles. The appearance of residues is also referred to as “flaking”. This is often observed with styling gels and is completely undesired.

It has now been found, in a manner which is surprising and unforeseeable by the person skilled in the art, that gel-like styling agents comprising one or more pregelatinized, crosslinked starch derivatives, anionic polymers, nonionic polymers, and water overcome the shortcomings of the prior art. By virtue of the described combination of modified starch (structure XL) with thickeners, it is possible to formulate gels with which very good setting of the hair is achieved and at the same time the formation of visible residues in the form of white particles or flakes on the hair upon removing the product by combing out can be reduced significantly.

The invention also covers the use of one or more pregelatinized, crosslinked starch derivatives for improving the ability of aqueous styling agents containing anionic polymers to be removed by combing the product out of the hair.

Here, it is particularly preferred if the pregelatinized, crosslinked starch derivatives used are hydroxypropylated phosphate esters, very particularly preferably hydroxypropyl distarch phosphates.

Preference is given to styling agents or uses according to the invention characterized in that the anionic polymer used is at least one substance chosen from the group of polyacrylic acids with a molar mass of from 2,000,000 g/mol to 6,000,000 g/mol, acrylic acid/acrylamide copolymer sodium salts with a molar mass of from 2,000,000 g/mol to 6,000,000 g/mol, acrylic acid/methacrylic acid copolymers, acrylate/steareth-2 methacrylate copolymer, steareth-10 allyl ether/acrylate copolymer, acrylate/steareth-20 itaconate copolymer, celluloses, cellulose derivatives, cellulose gums, ethoxylated celluloses, starch or gums, guar gum, guar hydroxypropyltrimonium chloride, xanthan gum, hydroxypropyl guar, karaya gum, bentonites, hectorites, polyquaternium-31 or polyquaternium-27, and the nonionic polymer used is at least one substance chosen from the group consisting of vinylpyrrolidone homo- or copolymers, preferably polyvinylpyrrolidone, N-vinylpyrrolidone/vinyl propionate copolymers, N-vinylpyrrolidone/vinyl acetate copolymers, polyvinylcaprolactam, polyvinylamides, vinylpyrrolidone/dimethylaminomethacrylate copolymers, vinylcaprolactam/vinylpyrrolidone/dimethylaminomethacrylate terpolymers, and polysiloxanes.

Particular preference is given to styling agents or uses according to the invention characterized in that the content of pregelatinized, crosslinked starch derivatives is 0.1 to 4% by weight, particularly preferably 1 to 4% by weight.

Very particular preference is given to styling agents or uses according to the invention characterized in that their viscosity is 7 to 13 Pa·s, preferably 9 to 11 Pa·s.

Although U.S. Pat. No. 6,248,338 describes cosmetic preparations with pregelatinized, crosslinked starch derivatives, this specification was unable to point the way to the present invention.

According to the invention, it is advantageous if the pregelatinized, crosslinked starch derivatives used are hydroxypropylated phosphate esters. Of particular advantage are those starch derivatives as described in U.S. Pat. No. 6,248,338, particularly advantageously hydroxypropyl distarch phosphate. Very particular preference here is given to the use of a hydroxypropyl distarch phosphate, as is sold in the form of the product Structure® XL by National Starch.

For the purposes of the present invention, preparations may advantageously be in the form of setting liquids or solutions or styling gels.

The compositions according to the invention can, for example, be in the form of preparations which are removable from squeezable bottles or via a pump device, but also in the form of a composition which can be applied from normal bottles and containers.

Besides an effective content of active ingredient combinations according to the invention, preparations according to the invention advantageously also comprise customary active substances, ingredients, additives and/or auxiliaries.

Cosmetic preparations according to the invention for setting and styling hair usually comprise film formers, as are normally used in such preparations, the total amount of the film former substances being, for example, between 0.5 and 20% by weight, based on the total weight of the preparations.

According to the invention, favorable film formers which may be used are all film formers customary or suitable for cosmetic and/or dermatological applications.

Depending on the preparation, the film former or formers are advantageously chosen from the group of alcohol- or water-soluble or dispersible polyurethanes, polyureas, silicone resins and/or polyesters, and nonionic, anionic, amphoteric and/or cationic polymers.

Advantageous nonionic polymers which may be present in preparations according to the invention alone or in a mixture, preferably also with anionic and/or amphoteric and/or zwitterionic polymers, are vinylpyrrolidone homo- or copolymers. These include, for example, polyvinylpyrrolidone, copolymers of N-vinylpyrrolidone and vinyl acetate and/or vinyl propionate in various concentration ratios, polyvinylcaprolactam, polyvinylamides and salts thereof, and copolymers of vinylpyrrolidone and dimethylaminoethyl methacrylate, terpolymers of vinylcaprolactam, vinylpyrrolidone and dimethylaminoethyl methacrylate, polysiloxanes and the like.

Advantageous anionic polymers are, for example, vinyl acetate/crotonic acid, vinyl acetate/acrylate and/or vinyl acetate/vinyl neodecanoate/crotonic acid copolymers, sodium acrylate/vinyl alcohol copolymers, sodium polystyrenesulfonate, ethyl acrylate/-N-tert-butylacrylamide/acrylic acid copolymers, vinylpyrrolidone/vinyl acetate/itaconic acid copolymers, acrylic acid/acrylamide copolymers and/or sodium salts thereof, homo- and/or copolymers of acrylic acid and/or methacrylic acid and/or salts thereof, and acrylate/hydroxyacrylate, octylacrylamide/acrylate or methacrylate and/or butyl acrylate/N-vinylpyrrolidone copolymers or polystyrenesulfonates.

Further preferred anionic polymers are methyl vinyl ether/maleic acid copolymers which are produced by hydrolysis of vinyl ether/maleic anhydride copolymers. These polymers may also be partially esterified (ethyl, isopropyl and butyl esters).

Advantageous amphoteric polymers which may be present in preparations according to the invention alone or in a mixture, preferably also with anionic and/or nonionic polymers, are copolymers of N-octylacrylamide, (meth)acrylic acid and tert-butylaminoethyl methacrylate of the “Amphomer” type, copolymers of methacryloylethylbetaine and alkyl methacrylates of the “Yukaformer” type, copolymers of monomers containing carboxyl groups or sulfone groups, e.g. (meth)acrylic acid and itaconic acid, with basic, in particular amino group-containing monomers, such as, for example, mono- or dialkylaminoalkyl (meth)acrylates and/or mono- and dialkylaminoalkyl (meth)acrylamides, copolymers of N-octylacrylamide, methyl methacrylate, hydroxypropyl methacrylate, N-tert-butylaminoethyl methacrylate and acrylic acid, where this list is of course not intended to be limiting.

It is in some cases advantageous to neutralize the anionic and amphoteric polymers with suitable bases to improve their compatibility, solubility or dispersibility with water or propellants. For this purpose it is possible to use, for example, alkali metal or alkaline earth metal bases, ammonia and/or various amines, such as, for example, triethanolamine, triisopropanolamine, aminomethylpropanol and/or aminomethylpropanediol. The neutralization may be partial or complete depending on the intended use.

Advantageous cationic polymers are, for example, vinylpyrrolidone/vinylimidazolium methochloride copolymers, quaternized vinylpyrrolidone/dialkylaminoalkyl methacrylate copolymers, cationic cellulose derivatives, such as, for example, hydroxyethylcellulose/dimethylalkylammonium chloride copolymers, and terpolymers of vinylcaprolactam/vinylpyrrolidone with dimethylaminoethyl methacrylate or vinylimidazolinium methochloride and acrylamido copolymers.

It is also advantageous to use film formers on a natural basis, e.g. chitosan and derivatives thereof, in particular in a mixture with synthetic polymers.

The preparations according to the invention may comprise cosmetic auxiliaries as are customarily used in such preparations, e.g. perfumes, preservatives, substances for reducing or stabilizing foam, dyes, pigments which have a coloring effect, surface-active substances, solubility promoters, thickeners, emulsifiers, complexing agents, sequestering agents, pearlizing agents, softening, moisturizing and/or humectant substances, refatting agents, alcohols, polyols and toxicologically compatible ethers and esters thereof, branched and/or unbranched hydrocarbons, further antioxidants, stabilizers, pH regulators, bodying agents, bactericides, deodorants, antimicrobial substances, antistats, UV absorbers, polymers, electrolytes, organic solvents, silicone derivatives, plant extracts, vitamins and/or other active ingredients or other customary constituents of a cosmetic or dermatological formulation.

The total amount of the auxiliaries is, for example, 0.001 to 15% by weight, preferably 0.01 to 10% by weight, in each case based on the total weight of the preparation.

The preparations according to the invention optionally advantageously comprise alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and in particular one or more thickeners which can be chosen advantageously from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

Gels according to the invention usually comprise alcohols of low carbon number, e.g. ethanol, isopropanol, 1,2-propanediol, glycerol and water or an abovementioned oil in the presence of a thickener which, in the case of oily-alcoholic gels, is preferably silicon dioxide or an aluminum silicate, and in the case of aqueous-alcoholic or alcoholic gels, is preferably a polyacrylate.

In the preparations, the content of thickeners is 0.01 to 20% by weight, preferably 0.01 to 10% by weight, in particular 0.05 to 5% by weight, in each case based on the total weight of the preparation. Here, the water content of the preparations is 50 to 95% by weight, preferably 60 to 95% by weight, in particular 70 to 95% by weight, in each case based on the total weight of the preparation. The content of alcohols in the aqueous preparations is, for example, 0 to 30% by weight, preferably 0 to 20% by weight, in particular 0 to 15% by weight, in each case based on the total weight of the preparation. The preparations according to the invention can comprise water, e.g. in the amounts introduced by the raw materials used, for example or e.g. also in amounts of up to 30% by weight, based on the total weight of the preparations.

Antioxidants which may be used according to the invention are all antioxidants customary or suitable for cosmetic and/or dermatological applications.

The total amount of the antioxidants is, for example, 0.001 to 2% by weight, preferably 0.01 to 1% by weight, in each case based on the total weight of the preparation.

Further antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-camosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, camosine, butylhydroxytoluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of the specified active ingredients which are suitable according to the invention.

Preparations according to the invention may advantageously also comprise substances which absorb UV radiation in the UV-B region, where the total amount of the filter substances is, for example, 0.001% by weight to 30% by weight, preferably 0.05 to 10% by weight, in particular 0.1 to 1.0% by weight, based on the total weight of the preparations, in order to provide cosmetic preparations which protect the hair and/or the skin from the entire range of ultraviolet radiation. They can also serve as sunscreens for the hair or the skin, in particular the scalp.

If the emulsions according to the invention comprise UV-B filter substances, these may advantageously be water-soluble. Advantageous water-soluble UV-B filters are, for example:
salts of 2-phenylbenzimidazole-5-sulfonic acid, such as its sodium, potassium or its triethanolammonium salt, and the 2-phenylbenzimidazole-5-sulfonic acid itself;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3-bomylidenemethyl)sulfonic acid and its salts.

It may also be advantageous to admix preparations according to the invention with UV-A filters which are hitherto customarily present in cosmetic preparations. It is possible to use the amounts used for the UV-B combination.

In the styling formulations according to the invention, the film formers which may be used are preferably nonionic or amphoteric polymers, such as, for example, PVPNA copolymers, which are available, for example, from BASF under the trade name Luviskol VA 64W or from International Speciality Products (ISP) under the name PVP/VA E 335, or nonionic polymers of the PVP/VA copolymer type, which are available under the trade name Luviskol VA 64W from BASF or under the name PVP/VA W735 from International Speciality Products (ISP), in concentrations of 2-8% by weight of the total weight of the preparation (based on the active content of the polymer).

Amphoteric polymers of the octylacrylamide/acrylates/butylaminoethyl methacrylate copolymer type are available, for example, from National Starch under the trade names Amphomer 28-4910 and LV-71 and are used, for example, in concentrations between 1-6% by weight of the total weight of the preparation.

Preference is also given to using cationic polymers of the polyquaternium-11 type (trade name Gafquat 755N, International Speciality Products (ISP)) or −16 (trade name Luviquat FC 550, BASF), which besides having setting properties also have conditioning properties and thus additionally act as care substances.

In the styling formulations based on alcohol according to the invention, care substances which may be used are preferably cyclic polydimethylsiloxanes (cyclomethicones) in concentrations of e.g. 0.1-1.0% by weight of the total formulation or silicone surfactants (polyether-modified siloxanes) of the dimethicone copolyol type, e.g. in concentrations of 0.01-1.0% by weight of the total weight of the preparation.

Cyclomethicones are supplied, inter alia, under the trade names Abil K4 from Goldschmidt or e.g. DC 244, DC 245 or DC 345 from Dow Corning. Dimethicone copolyols are supplied, for example, under the trade name DC193 from Dow Corning or Belsil DM 6031 from Wacker.

In the aqueous styling formulations according to the invention, e.g. in styling gels, instead of thickeners based on polyacrylic acid, it is also possible preferably to use silicone surfactants (polyether-modified siloxanes) of the dimethicone copolyol type as an alternative to the quats. Dimethicone copolyols are supplied, for example, under the trade name DC 193 by Dow Corning and Abil B 8851 by Goldschmidt. The use concentrations are, for example, between 0.02 and 1.0% by weight of the overall formulation.

It may in some instances be advantageous according to the invention to add complexing agents to the cosmetic or dermatological preparations used according to the invention.

Complexing agents are auxiliaries of cosmetology or of medicinal galenics known per se. The complexation of troublesome metals, such as Mn, Fe, Cu and others, may, for example, prevent undesired chemical reactions in cosmetic or dermatological preparations.

Complexing agents, in particular chelating agents, form complexes with metal atoms, which complexes represent metallacycles if one or more polybasic complexing agents, i.e. chelating agents, are present. Chelates are compounds in which a single ligand occupies more than one coordination site on a central atom. In this case, normally extended compounds are thus closed as a result of complex formation via a metal atom or metal ion to form rings. The number of bonded ligands depends on the coordination number of the central metal. A prerequisite for the formation of the chelate is that the compound reacting with the metal contains two or more atomic groups which act as electron donors.

The complexing agent or agents can advantageously be chosen from the group of customary compounds, preferably at least one substance from the group consisting of tartaric acid and anions thereof, citric acid and anions thereof, aminopolycarboxylic acids and anions thereof (such as, for example, ethylenediaminetetraacetic acid (EDTA) and anions thereof, nitrilotriacetic acid (NTA) and anions thereof, hydroxyethylene-diaminotriacetic acid (HOEDTA) and anions thereof, diethyleneaminopentaacetic acid (DPTA) and anions thereof, trans-1,2-diaminocyclohexanetetraacetic acid (CDTA) and anions thereof).

According to the invention, the complexing agent or agents are advantageously present in cosmetic or dermatological preparations preferably in amounts of from 0.01% by weight to 10% by weight, preferably in an amounts of 0.05% to 5% by weight, particularly preferably in amounts of 0.1-2.0% by weight, based on the total weight of the preparation.

The preparations according to the invention can be prepared in the customary manner by mixing the individual constituents. The active ingredients of the combinations according to the invention or else the premixed constituents of the combinations according to the invention may be added in the mixing operation.

The pH of the preparations can be adjusted in a known manner by adding acids or bases, preferably by adding buffer mixtures, e.g. based on citric acid/citrate or phosphoric acid phosphate buffer mixtures. Preferably, the pH is below 10, e.g. in the range from 3 to 9.

Unless stated otherwise, all amounts, fractions and percentages are based on the weight and the total amount or on the total weight of the preparations or of the particular mixture.

The examples below illustrate the invention.

The PVP/VA copolymer used in the examples is Luviskol VA 37 E (BASF). The octylacrylamide/acrylates/butylaminoethyl methacrylate copolymer used in the examples is Amphomer 28-4910 (National Starch). The amounts given in the examples are percentages by weight.

### EXAMPLES 1-2

| Gels | 1 | 2 |
|---|---|---|
| PVP/VA copolymer | 5.0 | 5.0 |
| Carbomer | 0.7 | 1.0 |
| Perfume, solubility promoter, care substances | q.s. | q.s. |
| Neutralization/pH adjustment | q.s. | q.s. |
| Ethanol | 10.0 | — |
| Hydroxypropyl starch phosphate ester (structure XL) | 0.3 | 0.2 |
| Water | 10.0 | 10.0 |

1. A gel-type styling agent preparation comprising
(a) one or more pregelatinized, crosslinked starch derivatives;
(b) one or more anionic polymers;
(c) one or more nonionic polymers; and
(d) water.

2. (canceled)

3. The preparation of claim 1, wherein the one or more pregelatinized, crosslinked starch derivatives include a hydroxypropylated phosphate ester.

4. The preparation of claim 3, wherein the one or more hydroxypropylated phosphate ester is hydroxypropyl distarch phosphate.

5. The preparation of claim 1, wherein the anionic polymer includes at least one substance selected from the group consisting of polyacrylic acids with a molar mass of 2,000,000 g/mol to 6,000,000 g/mol, acrylic acid/acrylamide copolymer sodium salts with a molar mass of from 2,000,000 g/mol to 6,000,000 g/mol, acrylic acid/methacrylic acid copolymers, acrylate/steareth-2 methacrylate copolymer, steareth-10 allyl ether/acrylate copolymer, acrylate/steareth-20 itaconate copolymer, celluloses, cellulose derivatives, cellulose gums, ethoxylated celluloses, starches, gums, guar gum, guar hydroxypropyltrimonium chloride, xanthan gum, hydroxypropyl guar, karaya gum, bentonites, hectorites, polyquaternium-31 and polyquaternium-27.

6. The that preparation of claim 1, wherein the content of pregelatinized, crosslinked starch derivatives is 0.1% to 4% by weight.

7. The preparation of claim 1, wherein the preparation has a viscosity of 7 to 13 Pa·s.

8. The preparation of claim 7, wherein the preparation has a viscosity of 9 Pa·s to 11 Pa·s.

9. The preparation of claim 6, wherein the content of pregelatinized, crosslinked starch derivatives is 1% to 4% by weight.

10. The preparation of claim 1, wherein the nonionic polymer includes at least one substance chosen from the group consisting of vinylpyrrolidone homo- or copolymers, N-vinylpyrrolidone/vinyl propionate copolymers, N-vinylpyrrolidone/vinyl acetate copolymers, polyvinylcaprolactam, polyvinylamides, vinylpyrrolidone/dimethylaminomethacrylate copolymers, vinylcaprolactam/vinylpyrrolidone/dimethylaminomethacrylate terpolymers, and polysiloxanes.

11. The preparation of claim 10, wherein the nonionic polymer includes polyvinylpyrrolidone.

12. A method for improving the ability of aqueous styling agents containing anionic polymers to be removed by combing the product out of the hair, said method comprising preparing said styling agents to include one or more pregelatinized, crosslinked starch derivatives.

13. The method of claim 12, wherein the one or more pregelatinized, crosslinked starch derivatives include a hydroxypropylated phosphate ester.

14. The method of claim 13, wherein the hydroxypropylated phosphate ester is hydroxypropyl distarch phosphate.

15. The method of claim 12, wherein the pregelatinized, crosslinked starch derivatives comprise 0.1% to 4% by weight of the styling agents.

16. The method of claim 15, wherein the pregelatinized, crosslinked starch derivatives comprise 1% to 4% by weight of the styling agents.
